(19) United States
(12) Patent Application Publication
(10) Pub. No.: US 2026/0110595 A1
(43) Pub. Date: Apr. 23, 2026

(54) AUTOMATIC DETECTION METHOD AND DEVICE FOR DETECTING SUBSTANCE COMPOSITION IN VISCOUS MATERIAL

(71) Applicant: AKTIEBOLAGET SKF, GOTEBORG (SE)

(72) Inventors: Guangzhou DIAO, Shanghai (CN); Yu Xin ZHOU, Shanghai (CN); Gang CHENG, Shanghai (CN); Defeng LANG, Delft (NL)

(21) Appl. No.: 18/681,647

(22) PCT Filed: Aug. 9, 2022
(86) PCT No.: PCT/EP2022/072347

§ 371 Date: Feb. 6, 2024

(30) Foreign Application Priority Data
Aug. 13, 2021 (CN) 202110932636.X

(51) Int. Cl. G01M 13/04; G01N 27/22; G01N 33/28

(52) U.S. Cl. CPC G01M 13/04; G01N 27/223; G01N 33/2847; G01N 33/2858

(57) ABSTRACT

An automatic detection method and an automatic detection device for detecting a substance composition in a viscous material are disclosed. The automatic detection method includes: detecting the viscous material in a preset detection period, obtaining a signal detection value corresponding to the viscous material, and generating a signal detection waveform; determining a detection state corresponding to the signal detection waveform according to the signal detection waveform, including a normal detection state and an abnormal detection state; and determining a content of the substance composition in the viscous material based on the signal detection waveform if the detection state is a normal detection state.

### TECHNICAL FIELD

Embodiments of the present disclosure relate to the field of detection, in particular to an automatic detection method and an automatic detection device for detecting substance composition in a viscous material.

### BACKGROUND

With the wide application of detection technology in civil and commercial fields, higher requirements are put forward for substance composition detection, especially for the substance composition detection of viscous materials.

At present, when testing a substance composition (such as a content of water or metal, etc.) included in a viscous material (such as grease, gel, etc.), it is usually done by manual measurement, by manual operations of related measurements and calculations to obtain the content of the related substance composition in the viscous material. However, on the one hand, the manual measurement leads to high measurement cost, poor measurement efficiency and low measurement speed, and the accuracy of the measurement results is greatly affected by manually operating steps, which may lead to considerable errors and low measurement reliability due to mis-operation. On the other hand, the scalability and adaptability of manual measurement are poor. In some industrial application scenarios, due to restrictions of application environment and coating position of the viscous material, the manual measurement process cannot be carried out, so that the detection and subsequent analysis of the substance composition in the corresponding viscous material cannot be realized.

Therefore, there is a need for a detection method with high detection reliability and efficiency, which can perform the detection in an automatic way on the premise of well detecting a substance composition in the viscous material and which can be well adapted to various industrial application scenarios.

### SUMMARY

In view of the above problem(s), the present disclosure provides an automatic detection method and an automatic detection device for detecting a substance composition in a viscous material. By utilizing the automatic detection method and the automatic detection device provided by the present disclosure, the detection process can be carried out in an automatic way on the basis of realizing good detection of the substance composition in the viscous material. The automatic detection method and the automatic detection device achieve high detection reliability and detection efficiency, have good expansibility, and can be adapted to various industrial application scenarios.

According to an aspect of the present disclosure, it provides an automatic detection method for detecting a substance composition in a viscous material, including: detecting the viscous material in a preset detection period, obtaining a signal detection value corresponding to the viscous material, and generating a signal detection waveform; determining a detection state corresponding to the signal detection waveform according to the signal detection waveform, the detection state including a normal detection state and an abnormal detection state; and determining a content of the substance composition in the viscous material based on the signal detection waveform if the detection state is the normal detection state.

In some embodiments, the detecting the viscous material in a preset detection period, obtaining a signal detection value corresponding to the viscous material and generating a signal detection waveform includes: performing a detection at a preset detection interval in the preset detection period by using a sensor detection circuit to obtain a plurality of signal detection values corresponding to the viscous material; and performing a fitting process based on the plurality of signal detection values to obtain the signal detection waveform.

In some embodiments, the determining a detection state corresponding to the signal detection waveform according to the signal detection waveform includes: determining the number of valley(s) in the signal detection waveform; and determining the detection state based on the number of valley(s).

In some embodiments, the determining the detection state based on the number of valley(s) includes: in the case where only a single valley is existed in the signal detection waveform, determining that the detection state is the normal detection state; in the case where two valleys are existed in the signal detection waveform, calculating an absolute value of a difference between valley values of the two valleys, determining the detection state as the normal detection state if the absolute value of the difference is greater than or equal to a preset threshold, and determining the detection state as the abnormal detection state if the absolute value of the difference is less than the preset threshold; and in the case where three or more valleys are existed in the signal detection waveform, sorting the valley values of the valleys from smallest to largest to obtain a valley sequence, calculating an absolute value of a difference between a first valley and a second valley in the valley sequence, comparing the absolute value of the difference with a preset additional threshold, determining the detection state as the normal detection state if the absolute value of the difference is greater than or equal to the preset additional threshold, and determining the detection state as the abnormal detection state if the absolute value of the difference is less than the preset additional threshold.

In some embodiments, the automatic detection method further includes: determining a waiting interval between a next detection and a current detection according to the detection state.

In some embodiments, the waiting interval is a first waiting interval if the detection state is determined to be the abnormal detection state, and the waiting interval is a second waiting interval if the detection state is determined to be the normal detection state, and the first waiting interval is smaller than the second waiting interval.

In some embodiments, the viscous material is a viscous material arranged on a target component of a fan, and a sensor detection circuit receives the viscous material on the target component of the fan; in the first waiting interval, continuing to receive the viscous material by the sensor detection circuit; and in the second waiting interval, blowing the viscous material received by the sensor detection circuit away from the sensor detection circuit firstly, and then continuing to receive the viscous material.

In some embodiments, the determining a content of the substance composition in the viscous material based on the signal detection waveform if the detection state is the normal detection state includes: taking a valley with a smallest valley value in the signal detection waveform as a target valley, and determining a detection valley value based on a valley value of the target valley; determining a detection environment parameter of the viscous material, the detection environment parameter including at least one of a detection temperature and a detection humidity; and inputting the detection environment parameter and the detection valley value into a machine learning model, and processing the detection environment parameter and the detection valley value by the machine learning model to obtain the content of the substance composition in the viscous material.

In some embodiments, the determining a detection valley value based on a valley value of the target valley includes: acquiring a preset number of signal detection waveforms with the normal detection state; determining a valley with the smallest valley value in each of the preset number of signal detection waveforms as the target valley; and weighting and averaging the valley values of the target valleys of the preset number of signal detection waveforms to obtain the detection valley value.

According to another aspect of the present disclosure, it provides an automatic detection device for detecting a substance composition in a viscous material. The automatic detection device realizes a detection of the viscous material according to the automatic detection method for detecting a substance composition in the viscous material described in any of the above.

By utilizing the automatic detection method and the automatic detection device provided by the present disclosure, the detection process can be carried out in an automatic way on the basis of realizing good detection of the substance composition in the viscous material. The automatic detection method and the automatic detection device achieve high detection reliability and detection efficiency, have good expansibility, and can be adapted to various industrial application scenarios.

### BRIEF DESCRIPTION OF THE DRAWINGS

In order to more clearly illustrate the technical solutions of the embodiments of the disclosure, the drawings necessary for the explanations of the embodiments will be briefly described in the following. It is obvious that the described drawings are only related to some embodiments of the disclosure, based on which other drawings may be obtained by those ordinary skilled in the art without paying any creative labors. The following drawings are not intended to be drawn to actual scales, with the focus on showing the main concept(s) of the present disclosure. In the drawings:

FIG. 1 shows an exemplary flowchart of an automatic detection method 100 according to an embodiment of the present disclosure;

FIG. 2 shows an exemplary flowchart of a process S101 of generating a signal detection waveform according to an embodiment of the present disclosure;

FIG. 3 shows an exemplary flowchart of a process S102 of determining a detection state corresponding to a signal detection waveform according to an embodiment of the present disclosure;

FIG. 4 shows, for example, an exemplary flowchart of a discrimination rule for determining a detection state based on the number of valleys;

FIG. 5 shows a schematic diagram of a signal detection waveform with more than three valleys according to an embodiment of the present disclosure;

FIG. 6 shows an exemplary process diagram of a process S103 of determining a content of substance composition in a viscous material according to an embodiment of the present disclosure;

FIG. 7 shows an exemplary flowchart of a grease detection process of a fan bearing by applying the automatic detection method according to an embodiment of the present disclosure; and

FIG. 8 shows a schematic diagram of a fan bearing and a sensor detection system.

### DETAILED DESCRIPTION

Hereinafter, technical solution(s) of the embodiments of the present disclosure will be described clearly and completely as below in conjunction with the accompanying drawings. It is apparent that the described embodiments are only a part of but not all of the embodiments of the present invention. Based on the described embodiments of the present invention, various other embodiments can be obtained by those of ordinary skill in the art without creative labor and those embodiments shall fall into the protection scope of the present invention.

As shown in the present application and claims, unless the context clearly indicates exceptional circumstances, words such as “a/an”, “one”, “one type of” and/or “the” are not specifically singular, but may also include plural. Generally speaking, the terms “comprising” and “including” only imply the inclusion of clearly identified steps and elements, but these steps and elements do not constitute an exclusive list, and methods or devices may also contain other steps or elements.

Although the present application makes various references to some modules in the system according to the embodiments of the present application, any number of different modules can be used and run on the user terminal and/or server. These modules are only illustrative, and different modules can be used for different aspects of the system and method.

A flowchart is used in the present application to explain the operations performed by the system according to the embodiment of the present application. It should be understood that, the preceding or following operations are not necessarily performed exactly in this order. On the contrary, various steps can be processed in reverse order or simultaneously, as required. At the same time, it can also add other operations to these procedures, or remove one or more operations from these procedures.

At present, when testing a substance composition (such as a content of water or metal, etc.) included in a viscous material (such as grease, gel, etc.), it is usually done by manual measurement, by manual operations of related measurements and calculations to obtain the content of the related substance composition in the viscous material. However, on the one hand, the manual measurement leads to high measurement cost, poor measurement efficiency and low measurement speed, and the accuracy of the measurement results is greatly affected by humanly operating steps, which may lead to considerable errors and low measurement reliability due to mis-operation. On the other hand, the scalability and adaptability of manual measurement are poor. In some industrial application scenarios, due to restrictions of application environment and coating position of the viscous material, the manual measurement process cannot be carried out, so that the detection and subsequent analysis of the substance composition in the corresponding viscous material cannot be realized.

Therefore, there is a need for a detection method with high detection reliability and efficiency, which can perform the detection in an automatic way on the premise of good detection of the substance composition in the viscous material and which can be well adapted to various industrial application scenarios.

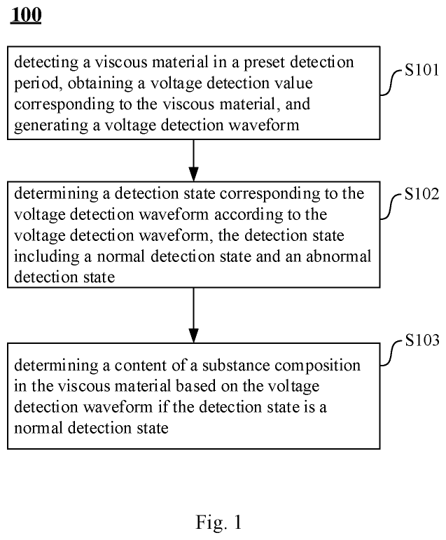

Based on the above, according to an aspect of the present disclosure, an automatic detection method for detecting a substance composition in a viscous material is provided. FIG. 1 shows an exemplary flowchart of an automatic detection method 100 according to an embodiment of the present disclosure.

Referring to FIG. 1, at first, in step S101, detecting a viscous material in a preset detection period, obtaining a signal detection value corresponding to the viscous material, and generating a signal detection waveform.

The viscous material refers to a material with a higher viscosity coefficient. The viscous material may include, for example, one or more of thick oil, grease, gel, emulsion and paste.

A substance composition in the viscous material refers to the composition of impurity substances in the viscous material. For example, according to different usage scenarios of the viscous material, the substance composition in the viscous material may include water, metal, etc.

It should be understood that the embodiments of the present disclosure are not limited by the specific substance composition of the viscous material and the specific types of substance composition to be detected in the viscous material.

The preset detection period can be selected by a user, for example, or can be determined according to the detection scenarios as applied and the requirements on the detection accuracy. According to actual needs, the preset detection period can be, for example, two minutes or ten minutes. The embodiment of the present disclosure is not limited by the specific setting mode and the duration set for the preset detection period.

In the preset detection period, the process of detecting the viscous material and obtaining the signal detection value corresponding to the viscous material can include, for example, performing a detection at a preset detection interval through a sensor detection circuit to obtain a plurality of signal detection values corresponding to the viscous material. Alternatively, other detection devices and methods can be adopted to obtain the signal detection value corresponding to the viscous material. The embodiment of the present disclosure is not limited by the specific way of obtaining the signal detection value corresponding to the viscous material.

The signal detection value refers to a signal value which corresponds to the viscous material to be detected and can reflect the property of the viscous material to be detected, e.g., a voltage value.

For example, when a sensor detection circuit is adopted for detection, the viscous material can be used as a capacitance component or a resistance component in the sensor circuit; a response signal value of the viscous material under the action of an input electric signal can be obtained by applying the input electric signal in the sensor detection circuit at a specific input frequency; and the response signal value can be used as a measurement signal value. However, it should be understood that, the embodiments of the present disclosure are not limited by the specific method of obtaining the signal detection value.

The process of generating the signal detection waveform based on the signal detection value can include, for example, continuously sampling a plurality of signal detection values corresponding to the viscous material in a preset detection period, and generating the signal detection waveform based on the plurality of signal values; alternatively, sampling signal detection values corresponding to the viscous material at equal intervals in the preset detection period, and then generating the signal detection waveform by a fitting process (such as an interpolation process) to the signal detection values. It should be understood that, the embodiments of the present disclosure are not limited by the specific way of generating the signal detection waveform.

After obtaining the signal detection waveform, determining a detection state corresponding to the signal detection waveform according to the signal detection waveform in step S102. The detection state includes a normal detection state and an abnormal detection state.

The detection state corresponding to the signal detection waveform refers to the information used for characterizing the reliability of the signal detection waveform as obtained. The normal detection state indicates that the signal detection waveform has relatively higher reliability and accuracy, and the signal detection waveform can well reflect the properties of the viscous material. The abnormal detection state indicates that the signal detection waveform has relatively lower reliability and accuracy, and the signal detection waveform cannot well reflect the properties of the viscous material.

It should be understood that, for example, the detection state of the signal detection waveform can be determined based on a waveform analysis of the signal detection waveform as obtained. For example, the detection state of the signal detection waveform can be obtained through the number of valleys in the signal detection waveform, by making a judgment based on a preset judgment rule; alternatively, the detection state of the signal detection waveform can be determined by comprehensively considering the characteristics of the signal detection waveform in both time domain and frequency domain. It should be understood that, the embodiments of the present disclosure are not limited by the specific way of determining the signal detection waveform.

For example, the detection state of the signal detection waveform can be characterized by a detection state signal. For example, the detection state of the signal detection waveform is characterized as a normal detection state by a detection state signal at a high level, and the detection state of the signal detection waveform is characterized as an abnormal detection state by a detection state signal at a low level. However, it should be understood that, according to actual needs, the detection state of the signal detection waveform can also be represented in other ways, such as binary coding or hexadecimal coding. The embodiment of the present disclosure is not limited by the specific representation of the detection state.

After determining the detection state corresponding to the signal detection waveform, determining a content of the substance composition in the viscous material based on the signal detection waveform if the detection state is a normal detection state in step S103.

The process of determining the content of substance composition in the viscous material based on the signal detection waveform can include, for example, determining a target valley based on the signal detection waveform, for example, taking a valley with the smallest valley value in the signal detection waveform as the target valley; and then determining a content value of the substance composition in the viscous material based on a processing operation by a machine learning model via the target valley and other measurement parameters (such as a detection environment parameter related to the detection process).

However, it should be understood that, the above only gives a way to determine the content of the substance composition in the viscous material. According to actual needs, for example, the signal detection waveform can be analyzed and processed by a preset algorithm or a specific processing function to obtain the content of the substance composition in the viscous material. The embodiment of the present disclosure is not limited by the specific way of determining the substance composition in the viscous material.

For example, the content of the substance composition in the viscous material can be determined only based on a single signal detection waveform in the normal detection state, or the content of the substance composition in the viscous material can be determined based on a plurality of signal detection waveforms in the normal detection state. The embodiment of the present disclosure is not limited by the specific number of signal detection waveform(s) as used.

Based on the above, in the present application, the viscous material is detected in a preset detection period, the signal detection value corresponding to the viscous material is obtained and the signal detection waveform is generated, and the detection state corresponding to the signal detection waveform is determined. When the detection state is a normal detection state, the content of the substance composition in the viscous material is determined based on the signal detection waveform. On the one hand, compared with the existing technical scheme of measuring the substance composition of the viscous material based on manual operations, the automatic detection method in the present application can perform the detection process in an automatic way without special manual operations, which significantly reduces the labor cost of measurement, improves the detection efficiency, and expands the application scenarios of the viscous material detection so that it can be well adapted to various industrial application scenarios (in which the viscous material is located in different coating positions and coating environments), thereby achieving reliable and accurate measurement of the substance composition in the viscous material. On the other hand, in the present application, after the signal detection waveform is obtained by measurement, the detection state of the signal detection waveform as measured is further judged, and only the signal detection waveform in the normal detection state (that is, the signal detection waveform that can well reflect the properties of the viscous material) is utilized to determine the substance composition in the viscous material, thus further improving the accuracy and reliability of the detection results.

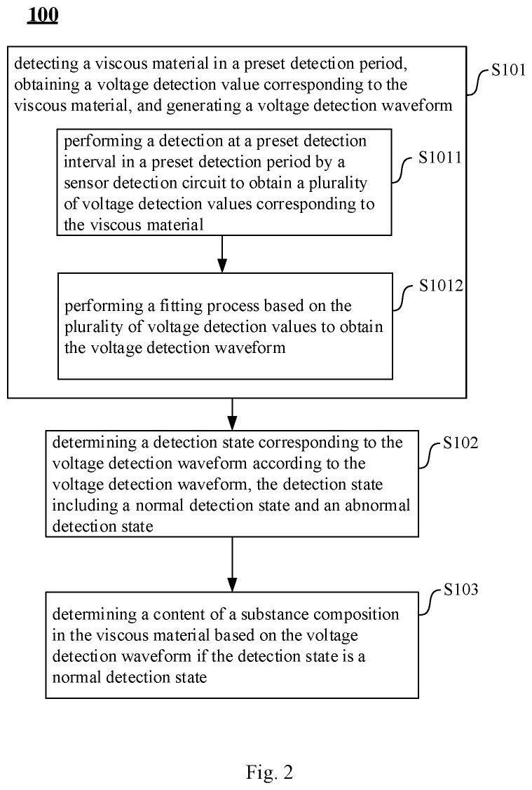

In some embodiments, the aforementioned process S101 of detecting the viscous material in a preset detection period, obtaining the signal detection value corresponding to the viscous material and generating the signal detection waveform can be described in more details, for example. FIG. 2 shows an exemplary flowchart of a process S101 of generating a signal detection waveform according to an embodiment of the present disclosure.

Referring to FIG. 2, at first, in step S1011, performing a detection at a preset detection interval in a preset detection period by a sensor detection circuit to obtain a plurality of signal detection values corresponding to the viscous material.

The sensor detection circuit refers to a detection device for determining signal values corresponding to the viscous material based on at least a part of the viscous material. The sensor detection circuit detects and obtains the signal detection values corresponding to the viscous material at preset, equal, detection intervals, for example.

The preset detection interval can be set by a user, for example, or can be determined by a sensor detection circuit based on actual needs. The preset detection interval may be, for example, 100 milliseconds or 1 second, and the embodiment of the present disclosure is not limited by the specific value of the preset detection interval and its setting mode.

For example, the detection process of the sensor detection circuit can be explained more specifically. For example, the sensor detection circuit can be arranged near the coating area of the viscous material and continuously receive the viscous material (for example, when the viscous material is coated on a motor shaft of the fan, the sensor detection circuit can be arranged below the motor shaft to receive the dripped viscous material), and the sensor detection circuit can, for example, apply an input signal with a specific frequency range (e.g., a sine wave signal with a frequency in the range of 2 kHz-3 kHz) to the viscous material (at this time, the viscous material is regarded as a resistance or capacitance component to be measured in the sensor detection circuit), so that the viscous material will generate a response signal in response to the input signal, and the sensor detection circuit will collect the response signal at the viscous material according to the preset detection interval and output a response signal value. The response signal value is regarded as the signal detection value corresponding to the viscous material. For example, if the preset detection period is 2 minutes and the preset detection interval of the sensor detection circuit is 1 second, the sensor detection circuit can detect and obtain 120 signal detection values corresponding to the viscous material in the detection period.

After obtaining the plurality of signal detection values, performing a fitting process based on the plurality of signal detection values to obtain the signal detection waveform in step S1012.

The fitting process includes: estimating signal detection values at one or more other time points in the preset detection period based on the plurality of signal detection values detected by the sensor detection circuit in the preset detection period (the plurality of signal detection values are discrete in time domain), generating fitting signal detection values, and generating the signal detection waveform based on the fitting signal detection values and the signal detection values together.

For example, one or more fitting signal detection values can be generated between two adjacent signal detection values by interpolation method, or, other methods can be used for performing the fitting process to obtain a plurality of fitting signal detection values. The embodiment of the present disclosure is not limited by the specific mode of the fitting process.

For example, if the number of the discrete, signal detection values as collected is 100, for example, 900 fitting signal detection values at other time points in the preset detection period can be further generated through the fitting process. In such case, the 100 signal detection values and the 900 fitting signal detection values, together, are used as target signal values, and the target signal values can be further connected sequentially based on the time points in time domain to obtain the signal detection waveform.

Based on the above, in the present application, a plurality of signal detection values corresponding to the viscous material is obtained by the sensor detection circuit performing a detection at a preset detection interval in a preset detection period, so that the plurality of signal detection values corresponding to the viscous material can be efficiently and accurately measured by the sensor detection circuit; and a signal detection waveform is obtained by further performing a fitting process based on the plurality of signal detection values, in this way, on the one hand, the accuracy and reliability of the signal detection waveform as generated are effectively improved by estimating fitting signal detection values at other time points in the preset detection period based on the signal detection values and then generating the signal detection waveform through the fitting signal detection values and the signal detection values together; and on the other hand, the number of the signal detection values to be detected by the sensor detection circuit can also be significantly reduced through the fitting process, so that the amount of detection data at the sensor detection circuit can be decreased and the working efficiency of the sensor detection circuit can be improved.

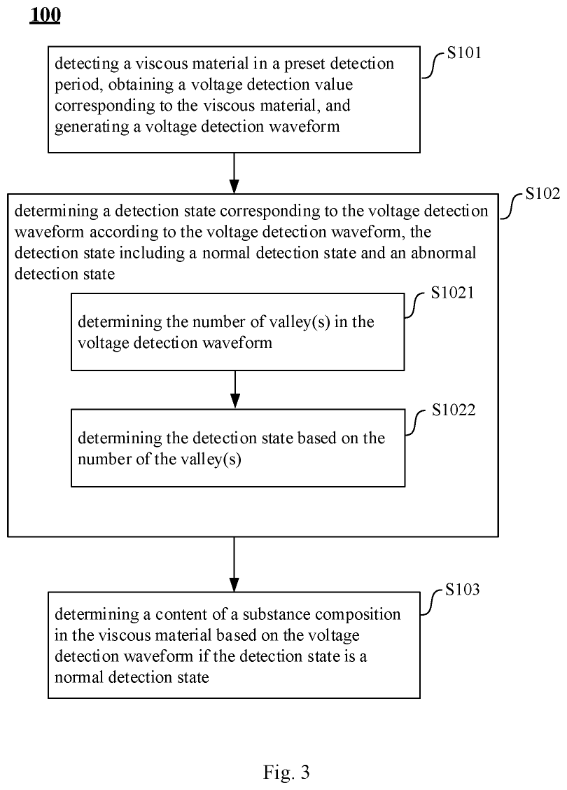

In some embodiments, the aforementioned process S102 of determining the detection state corresponding to the signal detection waveform according to the signal detection waveform can be described in more details, for example. FIG. 3 shows an exemplary flowchart of a process S102 of determining a detection state corresponding to a signal detection waveform according to an embodiment of the present disclosure.

Referring to FIG. 3, at first, in step S1021, determining the number of valley(s) in the signal detection waveform.

The valley can be more specifically explained, for example. If a signal value of a certain point in the signal detection waveform is the smallest as compared with the signal values of its nearby points, the certain point becomes the valley of the signal detection waveform, and the signal value corresponding to the certain point is the valley value of the valley.

It should be understood that, the valley is only used to characterize the signal minimum point existed in the signal detection waveform. According to actual situations, there may be one or more minimum points in the signal detection waveform, that is, there may be one or more valleys.

Thereafter, in step S1022, determining a detection state based on the number of the valley(s).

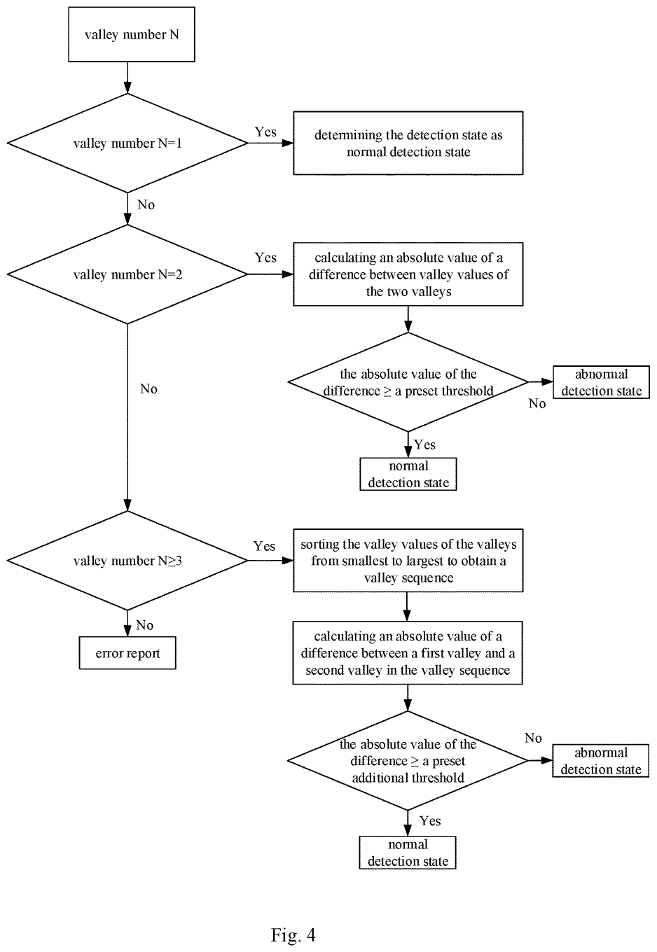

For example, different rules can be adopted to determine the detection state, according to the difference in the number of valley(s) existed in the current signal detection waveform. For example, different discrimination rules can be flexibly set according to different situations in which the signal detection waveform has single, two, three or more valleys (examples of specific discrimination rules will be shown in details below with reference to FIG. 4), so as to determine the detection state.

Based on the above, in the present application, in the process of determining the detection state corresponding to the signal detection waveform, the number of valley(s) in the signal detection waveform is determined and then the detection state is determined based on the number of valley(s), so that the judgment of the detection state can be achieved by flexibly setting different judgment rules according to actual needs for different number of valley(s) in the signal detection waveform. Compared with the situation that the detection state is judged through detailed analysis of the signal detection waveform or real-time monitoring of the whole process of sensor detection, the detection state of the present application is determined only through the number of the valley(s), which simplifies the determination process of the detection state and improves the efficiency and speed of automatic detection.

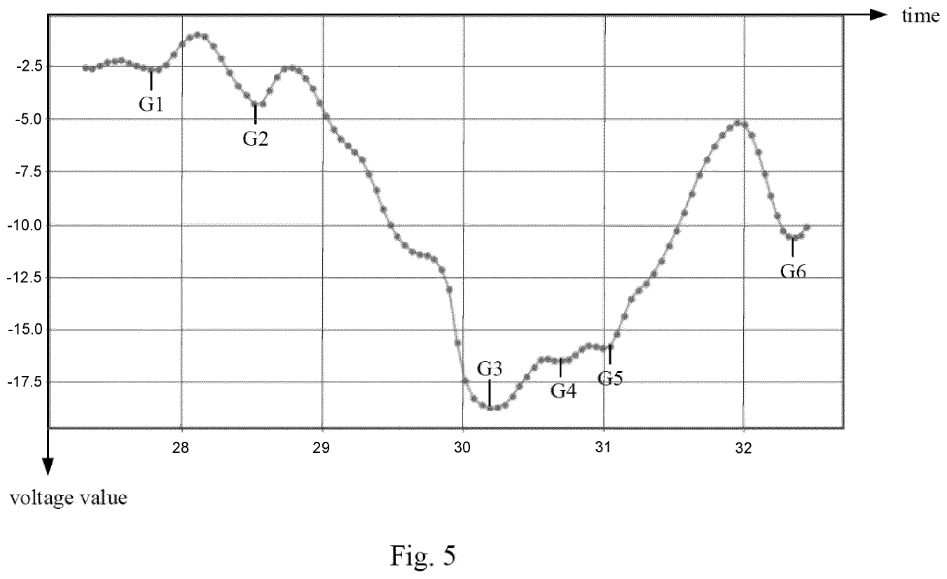

FIG. 4 shows, for example, an exemplary flow chart of a discrimination rule for determining a detection state based on the number of valley(s). FIG. 5 shows a schematic diagram of a signal detection waveform with more than three valleys according to an embodiment of the present disclosure. Hereinafter, the above discrimination rule will be explained in more details with reference to FIGS. 4 and 5.

For example, as shown in FIG. 4, when the number of the valley(s) in the signal detection waveform is one, two, three or more, different discrimination rules can be set respectively.

Among them, after obtaining the valley number N of the signal detection waveform, when there is only a single valley (N=1) in the signal detection waveform, the detection state of the signal detection waveform can be determined as the normal detection state, for example.

When there are two valleys (N=2) in the signal detection waveform, an absolute value of a difference between valley values of the two valleys (that is, the absolute value of the difference between the two valleys) can be calculated and compared with a preset threshold. When the absolute value of the difference is greater than or equal to the preset threshold, determining that the detection state is a normal detection state; and when the absolute value of the difference is less than the preset threshold, determining that the detection state is an abnormal detection state.

It should be understood that, the preset threshold can be set according to actual needs, for example, and the embodiment of the present disclosure is not limited by the setting mode of the preset threshold and the specific value of the preset threshold as set.

When there are three or more valleys (N≥3) in the signal detection waveform, for example, the valley values of the valleys can be sorted from smallest to largest to obtain a valley sequence. Thereafter, an absolute value of a difference between a first valley and a second valley in the valley sequence is further calculated, and the detection state is determined based on the absolute value of the difference. For example, the detection state can be determined based on the comparison between the absolute value of the difference and a preset additional threshold, or the detection state can be determined according to the absolute value of the difference in other ways.

For example, the signal detection waveform shown in FIG. 5 has, for example, six valleys, which can be marked as valleys G1, G2, G3, G4, G5, and G6, respectively, in chronological order. In such case, according to the above discrimination rule, for example, the valley values of the six valleys can be sorted from smallest to largest, so as to obtain a valley sequence of G3, G4, G5, G6, G1 and G2. Then, for example, an absolute value of a difference between the first valley G3 and the second valley G4 in the valley sequence can be calculated. If the signal value of the first valley G3 is −19 and the signal value of the second valley G4 is −16.5, the absolute value of the difference between them is 2.5. Thereafter, referring to FIG. 4, determining the detection state based on the absolute value of the difference at this time by, for example, comparing the absolute value of the difference with a preset additional threshold; determining the detection state as a normal detection state if the absolute value of the difference is greater than or equal to the preset additional threshold; and determining the detection state as an abnormal detection state if the absolute value of the difference is less than the preset additional threshold. If the preset additional threshold is 3, the detection state of the signal detection waveform at this time is an abnormal detection state.

It should be understood that, the preset threshold and the preset additional threshold are only used to distinguish the threshold for two valleys from the threshold for three or more valleys, and are not intended to limit the preset threshold and the preset additional threshold. According to actual needs, the preset threshold and the preset additional threshold can be set to the same value, for example, or can be set to different values.

Based on the above, by distinguishing three situations respectively having one, two and three or more valleys, it is possible to set up targeted discrimination rules for signal detection waveforms with different number of valleys, so as to simply and efficiently determine the detection state; and at the same time, it is beneficial for simplifying the discrimination process and improving the detection speed.

In some embodiments, if the number of the valley(s) does not meet any of the above three situations (one valley, two valleys and three or more valleys), it is considered that there is a significant abnormality in the sensor detection process at this time, and then an alarm process can be executed, for example.

In some embodiments, determining the detection state corresponding to the signal detection waveform according to the signal detection waveform further includes: determining that the signal detection waveform is an abnormal detection waveform when the signal detection waveform is an initial detection waveform.

By determining the detection state of the initially detected signal detection waveform as an abnormal detection state, it can avoid the problem that there may be a big error in the determined content of substance composition in the viscous material if the sensor detection circuit has not been placed in a right place or well installed in the corresponding position yet at the time of the initial detection, or if the content of the viscous material received by the sensor detection circuit is too small, thereby improving the accuracy of the automatic detection process.

In some embodiments, the automatic detection method can be cyclically executed, for example, after the current detection is completed, the next detection is performed after a certain waiting interval.

By cyclically executing the automatic detection method, the content of substance composition in the viscous material can be detected in real time in the usage process of the viscous material, which is beneficial for timely analysis of the usage and the current situation of the viscous material for timely treatment.

In some embodiments, for example, according to the detection state, the waiting interval between the next detection and the current detection is determined. The waiting interval refers to a time interval between the completion of the current detection and the start of the next detection.

For example, the waiting interval when the detection state is determined as an abnormal detection state may be set to be smaller than the waiting interval when the detection state is determined as a normal detection state.

Based on the above, in the present application, by determining the waiting interval between the next detection and the current detection according to the detection state, it is beneficial for adjusting the waiting interval depending on different detection states, and hence is beneficial for the sensor detection circuit or related detection devices to perform different actions in the waiting interval according to actual needs, so as to adjust or to be better adapted to the detection scenario and realize the high-precision detection of the substance composition in the viscous material.

In some embodiments, the process of determining the waiting interval between the next detection and the current detection based on the detection state can be described in more details, for example. For example, when the detection state is determined as an abnormal detection state, the waiting interval is a first waiting interval, and when the detection state is determined as a normal detection state, the waiting interval is a second waiting interval; and the first waiting interval is smaller than the second waiting interval.

The first waiting interval may be 30 minutes, for example, and the second waiting interval may be 40 minutes. Alternatively, the first waiting interval may be 60 minutes, for example, and the second waiting interval may be 90 minutes.

By setting the waiting interval as the first waiting interval when the detection state is an abnormal detection state, and setting the waiting interval as the second waiting interval when the detection state is a normal detection state, and by setting the first waiting interval to be smaller than the second waiting interval, a longer waiting interval can be achieved when the detection state is a normal detection state, so that the sensor detection circuit or the detection device can perform more actions, such as cleaning the currently received viscous materials, etc., in this longer waiting interval, which is beneficial for improving the accuracy and working efficiency of the automatic detection method.

Hereinafter, different actions performed in the first waiting interval and the second waiting interval will be explained in more details in combination with specific embodiments. In some embodiments, the viscous material is a viscous material arranged on a target component of a fan, and the sensor detection circuit receives the viscous material on the target component of the fan.

The target component of the fan refers to a component used to realize a specific purpose and function of the fan. According to actual needs, the target component can be, for example, a fan bearing assembly of the fan, or other functional components of the fan that need to be coated with viscous materials. The embodiment of the present disclosure is not affected by the specific type and position of the target component.

For example, the sensor detection circuit can be arranged near the target component to receive the viscous material. For example, when the target component is a main shaft (arranged on a bearing seat) of the fan and the viscous material is a lubricating grease, the bearing seat is provided with an opening, for example, and the sensor detection circuit extends into the bearing seat through the opening and is arranged below the main shaft of the fan to receive the viscous material dropped along with the operation of the fan. Usually, the sensor detection circuit has to wait for a certain time until the volume of the dropped viscous material is large enough, for example, so that the viscous material can well cover the relevant detection surface of the sensor detection circuit.

In such case, when the detection state is determined as an abnormal detection state, the waiting interval is the first waiting interval, and the sensor detection circuit continues to receive the viscous material in the first waiting interval.

Because the detection state of the currently detected signal detection waveform is an abnormal detection state, it is characterized in that, in the current detection, the detected signal detection waveform can't well reflect the properties of the viscous material, then it is necessary to continue to detect the currently received viscous material by the sensor detection circuit. By arranging the sensor detection circuit to continue to receive the viscous material in the first waiting interval, the volume of the viscous material received by the sensor detection circuit can be continuously increased, so that the viscous material can well cover the relevant detection surface of the sensor detection circuit, and the detection accuracy in the next detection process can be improved.

When the detection state is determined as a normal detection state, the waiting interval is a second waiting interval larger than the first waiting interval, and in the second waiting interval, the viscous material received by the sensor detection circuit is blown away from the sensor detection circuit firstly, then the sensor detection circuit continues to receive the viscous material.

In such case, because the detection state of the signal detection waveform in the current detection process is a normal detection state, it's intended to represent that the signal detection waveform has high reliability and can well reflect the properties of the viscous material. Therefore, the viscous material currently received by the sensor detection circuit has been well detected. At this time, by blowing the viscous material (that is, the viscous material applied in the current detection process) received by the sensor detection circuit away from the sensor detection circuit, the detection surface of the sensor detection circuit is in a clean state, and then the sensor detection circuit continues to receive the newly dropped viscous material, so that the next detection process can be performed only on the newly dropped viscous material, and the viscous material having been well detected in the current detection will not be re-detected.

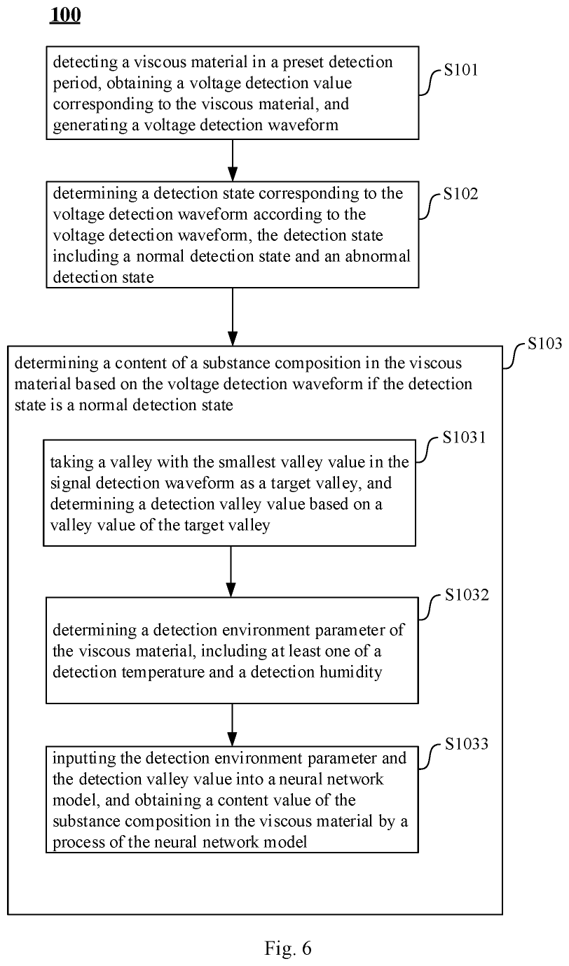

In some embodiments, when the detection state is a normal detection state, the process S103 of determining the content of the substance composition in the viscous material based on the signal detection waveform can be described in more details, for example. FIG. 6 shows an exemplary process diagram of a process S103 of determining a content of a substance composition in a viscous material according to an embodiment of the present disclosure.

When the detection state is a normal detection state, referring to FIG. 6, firstly, in step S1031, taking a valley with the smallest valley value in the signal detection waveform as a target valley, and determining a detection valley value based on a valley value of the target valley.

It should be understood that, the target valley is a valley in the signal detection waveform used for determining the detection wave value. By setting the valley with the smallest valley value in the signal detection waveform as the target valley, the target valley can well reflect the core characteristics of the signal detection waveform.

The detection valley value refers to a valley value used for determining the substance composition of the viscous material. For example, the detection valley value can be determined only by a single target valley, for example, the valley value of the target valley is directly determined as the detection valley value. Alternatively, the detection valley value can be determined by a plurality of target valleys respectively from a plurality of signal detection waveforms. The embodiment of the present disclosure is not limited by the number of the target valley(s) required for determining the detection valley value.

Thereafter, in step S1032, determining a detection environment parameter of the viscous material, including at least one of a detection temperature and a detection humidity.

It should be understood that, the above steps S1031 and S1032 can be executed sequentially, in reverse order or in parallel, for example. The embodiment of the present disclosure is not limited by the specific execution sequence of the steps S1031 and S1032.

Thereafter, in step S1033, inputting the detection environment parameter and the detection valley value into a machine learning model, and obtaining a content value of the substance composition in the viscous material by a process of the machine learning model.

The machine learning model can be, for example, a neural network-based model or other machine learning models. The embodiment of the present disclosure is not limited by the specific machine learning type to which the machine learning model is applied.

For example, the detection environment parameter and the detection valley value can be input to an input end of the machine learning model, and processed by the machine learning model; and the content of the substance composition in the viscous material corresponding to the detection environment parameter and the detection valley value can be output at an output end of the machine learning model. For example, when detecting the contents of water and iron in viscous oil, for example, the percentage of water in the viscous oil and the percentage of iron in the viscous oil can be obtained at the output end of the machine learning model.

A training process of machine learning can be described more specifically, for example. The training process includes, for example, data marking, model training and testing.

Firstly, in the process of data marking, for example, the detection can be performed for many times through changing the content of the substance composition in the viscous material at each detection temperature and each detection humidity to obtain corresponding signal detection waveforms, and the detection valley values can be determined based on the signal detection waveforms, so as to determine a correspondence set between the content of the substance composition in the corresponding viscous material and a detection data set constituted by the detection valley values, the detection temperatures and the detection humidity degrees.

Thereafter, in the process of model training and testing, half of the correspondences in the correspondence set are used as training data, in which the input data is the detection data set (including the detection valley values, the detection temperatures and the detection humidity degrees), and the output target variable is the content of the substance composition in the viscous material. The other half of the correspondences in the correspondence set is used as testing data. A machine learning algorithm is applied to build the machine learning model (such as a regression model), and the machine learning model is trained by the training data, and the reliability and accuracy thereof are verified by the testing data. Thus, the machine learning model is obtained.

Based on the above, by determining the target valley and further determining the detection valley value based on the signal detection waveform, and by inputting the detection valley value and the detection environmental parameter into the machine learning model and obtaining the content value of the substance composition in the viscous material through processing the machine learning model, the content of the substance composition in the viscous material can be obtained in a simple and convenient way through machine learning via the signal detection waveform and the detection environmental parameter, thus realizing the automatic detection process and effectively improving the detection accuracy and speed.

In some embodiments, when a plurality of signal detection waveforms with a normal detection state is applied to determine the detection valley value, the above-mentioned process of determining the detection valley value based on the target valley can be explained more specifically, for example.

Firstly, a preset number of signal detection waveform(s) in a normal detection state can be obtained. The preset number can be specified by the user or set by the system, for example. For example, eight signal detection waveforms in a normal detection state corresponding to the viscous material can be obtained, or twenty signal detection waveforms in a normal detection state corresponding to the viscous material can also be obtained. The embodiment of the present disclosure is not limited by the specific value of the preset number and the setting mode thereof.

Thereafter, for each of the signal detection waveform(s), the valley with the smallest valley value in the signal detection waveform is determined as the target valley. Finally, the valley values of the target valleys of these signal detection waveforms are weighted and averaged to obtain the detection valley value.

For example, three signal detection waveforms W1, W2 and W3 in a normal detection state corresponding to the viscous material are obtained, and the target valleys corresponding to the three signal detection waveforms are respectively Gt1, Gt2 and Gt3. The valley value of the target valley Gt1 is, for example, −10V, the valley value of the target valley Gt2 is, for example, −12V, and the valley value of the target valley Gt3 is, for example, −8V. After weighting and averaging the valley values of the three target valleys, the detection valley value can be obtained as −10V.

Based on the above, in the present application, the valley values of the target valleys of a plurality of signal detection waveforms in a normal detection state are weighted and averaged to obtain the detection valley value. Compared with the case based on only a single signal detection waveform, this method reduces the influence of each signal detection waveform on the detection valley value, avoids the serious influence of an error in a single detection to the subsequent measurement, and further improves the accuracy of the detection valley value.

In some embodiments, the method further includes: after determining the detection valley value, uploading the detection valley value, the signal detection waveform in a normal detection state corresponding to the detection valley value, and the valley data in the signal detection waveform to a cloud platform.

The signal detection waveform in a normal detection state corresponding to the detection valley value refers to the signal detection waveform used for generating the detection valley value. According to actual situations, for example, there may be a single signal detection waveform, or a preset number of signal detection waveform(s).

The valley data in the signal detection waveform refers to the related data of each valley in the signal detection waveform, such as the time point at which the valley is located, and the valley value of the valley.

After determining the detection valley value, the detection valley value, the signal detection waveform in a normal detection state corresponding to the detection valley value and the valley data in the signal detection waveform are uploaded to the cloud platform. In this way, on the one hand, the data storage capacity of the detection terminal can be reduced, thereby optimizing the detection process and improving the detection speed; and on the other hand, it is beneficial for the real-time storage of the detection data of the viscous material, and hence is beneficial for the subsequent analysis and subsequent optimization based on the data.

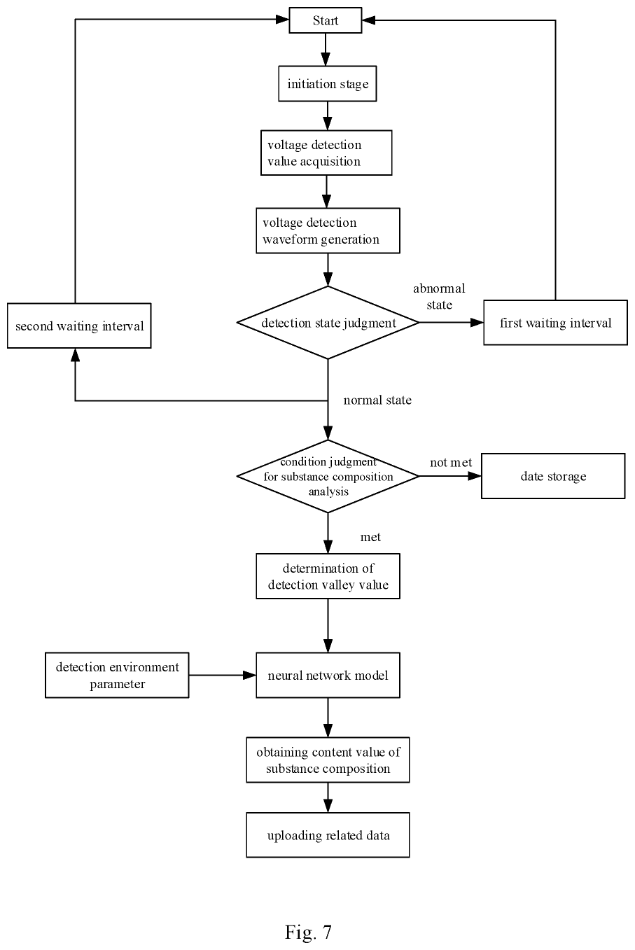

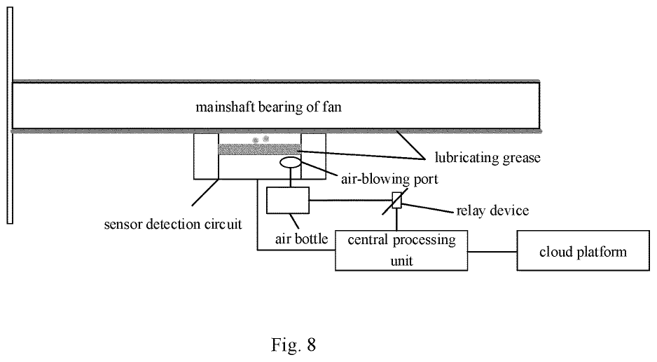

Hereinafter, the above-mentioned automatic detection method will be explained in combination with the application scenario of detecting a substance composition in the lubricating grease for fan bearings. FIG. 7 shows an exemplary flowchart of a grease detection process of a fan bearing by applying an automatic detection method according to an embodiment of the present disclosure. FIG. 8 shows a schematic diagram of a fan bearing and a sensor detection circuit.

Among them, for example, the above automatic detection method can be applied to realize a content detection of a deposition such as water and iron in the lubricating grease in inner and outer rings of a fan bearing by using an automatic detection system, so as to realize the judgment of the current running state and grease state of the fan, thereby replacing or adjusting the fan bearing in good time.

The automatic detection system can include, for example, a sensor detection circuit, a central processing unit and a cloud platform. Referring to FIG. 8, for example, the sensor detection circuit is arranged below the fan bearing to receive lubricating grease dropped along with the operation of the fan, or it can extend into the fan bearing.

In such case, first of all, for example, in an initialization stage, core detection parameters can be set in the central processing unit. The core detection parameters include, for example, a preset detection period, a preset detection interval, the number of fitting signal detection values to be generated by a fitting process, a first waiting interval and a second waiting interval. The meanings of the related parameters are as same as that mentioned above, and will not be repeated here.

In such case, for example, in an initialization process, the preset detection period can be set to 2 minutes, the preset detection interval can be set to 1 second, the number of fitting signal detection values to be generated by the fitting process can be set to 880, the first waiting interval can be set to 30 minutes and the second waiting interval can be set to 40 minutes, by the user or automatically by the system.

Thereafter, in a detection stage, for example, the viscous material can be detected by the sensor detection circuit in the preset detection interval, the signal detection value corresponding to the viscous material can be obtained, and the signal detection waveform can be generated, according to the step S101 in the automatic detection method 100 described above. At this time, for example, in a detection period of 2 minutes, the sensor detection circuit can perform a detection at a preset detection interval of 1 second, so as to obtain 120 signal detection values. Then, based on the plurality of signal detection values, a fitting process is performed to obtain 880 fitting signal detection values, and a signal detection waveform can be obtained based on the 120 signal detection values and the 880 fitting signal detection values (1000 signal values in total).

Thereafter, for example, the detection state corresponding to the signal detection waveform can be determined according to the signal detection waveform, according to the step S102 in the automatic detection method 100 described above. Specifically, for example, the detection state can be judged based on the number of the valley(s) in the signal detection waveform, for example, based on the aforementioned discrimination rule in FIG. 4, different situations with a single valley, two valleys, and three or more valleys are respectively processed to obtain the detection state of the signal detection waveform.

After the detection state is judged, if the detection state of the signal detection waveform is obtained as an abnormal detection state, it indicates that the signal detection waveform of this time of detection can't well reflect the properties of the lubricating grease. In such case, it will wait for a first waiting interval (30 minutes) before the next detection. During the first waiting interval, for example, as mentioned above, the sensor detection circuit will continue to receive the lubricating grease, so that the lubricating grease can better cover the relevant detection surface of the sensor detection circuit, and the accuracy of the next detection is improved.

Furthermore, when it is determined that the currently detected signal detection waveform is in a normal detection state after the detection state judgment, on the one hand, the sensor detection circuit will wait for a second waiting interval (40 minutes) before the next detection, and during the second detection interval, for example, the sensor detection circuit will blow the viscous material received by the sensor detection circuit away from the sensor detection circuit (the first 10 minutes) and then continue to receive the viscous material (the last 30 minutes), and will not re-detect the lubricating grease which has been well detected. Specifically, for example, referring to FIG. 8 showing an air-blowing device, i.e., an air bottle, the air bottle blows air through an air-blowing port and is controlled by the central processing unit via a relay device. Specifically, for example, in the second waiting time interval, based on a control instruction of the central processing unit, the relay device is turned on, so that the air bottle is powered to blow air, and the viscous material in the sensor detection circuit is blown away. When the air blow is continuously performed for 10 minutes, the central processing unit turns off the relay device, for example, so that the air bottle stops working and no longer blows air.

On the other hand, for example, the central processing unit can determine the content of the substance composition in the lubricating grease via the aforementioned step S103. At this time, according to the settings of the system, for example, it can judge the analysis condition of the substance composition analysis, firstly. For example, the settings for this time of detection include: determining the detection valley value based on eight signal detection waveforms in a normal detection state corresponding to the lubricating grease, and determining the content of the substance composition in the viscous material accordingly. In such case, for example, it can be judged whether eight signal detection waveforms in a normal detection state have been collected at present.

If eight signal detection waveforms in a normal detection state have not been collected yet at present, the analysis condition has not been met. At this time, for example, the central processing unit saves the current signal detection waveform and the related detection environment parameter (the detection environment parameter can be measured by the sensor detection circuit, for example). If eight signal detection waveforms in a normal detection state have been collected at present, the analysis condition has been met. At this time, for example, the detection valley value can be determined based on the eight signal detection waveforms in a normal detection state (the specific steps have been described in details in the foregoing, and will not be repeated here). Then, for example, the detection environment parameter of the viscous material can be determined, including the detection temperature and the detection humidity (for example, the average value of the detection temperatures and detection humidity degrees corresponding to the eight signal detection waveforms). Further, the detection environmental parameter and the detection valley value are inputted into the machine learning model, and are processed by the machine learning model to obtain the content value of the substance composition in the viscous material.

After the content values of water component and iron component in the lubricating grease are generated, for example, the detection valley value, the eight signal detection waveforms corresponding to the detection valley value, and the valley data (time points and valley values) in the eight signal detection waveforms can be uploaded to the cloud platform for real-time backup, and relevant data stored in the central processing unit can be deleted to save the storage space. Therefore, the real-time and high-precision automatic detection of the fan bearing can be realized.

According to another aspect of the present disclosure, an automatic detection device for detecting a substance composition in a viscous material is provided. The automatic detection device realizes the detection of viscous material according to the aforementioned automatic detection method for detecting a substance composition in a viscous material.

In some embodiments, the automatic detection device can receive the viscous material, and can perform the above-described automatic detection method and realize the above-described automatic detection function.

The program part of the technology can be considered as a “product” or an “article of manufacture” in the form of executable code(s) and/or related data, which is involved or realized by a computer-readable media. A tangible and permanent storage media can include any internal storage or memory used by a computer, a processor, or similar devices or related modules. For example, various semiconductor memories, tape drives, disk drives, or similar devices that can provide storage functions for software.

All or part of software may sometimes communicate through a network, such as the Internet or other communication networks. Such communication can load software from one computer device or processor to another. For example, loading from a server or host computer of a target tracking device to a hardware platform of a computer environment, or other computer environments that implement the system, or systems with similar functions related to providing required information. Therefore, another medium capable of transmitting software elements can also be used as a physical connection between local devices, such as light waves, electric waves, electromagnetic waves, etc., which can be spread through electrical cable, optical cable or air. The physical media for carrying the waves, such as electrical cable, wireless connection or optical cable, can also be considered as a media for carrying software. As used here, unless limited to the tangible “storage” media, other terms indicating computer or machine “readable media” all refer to the media involved in the execution of any instruction by the processor.

The present application uses specific words to describe the embodiments of the present application. Words such as “the first/second embodiment”, “one embodiment” and/or “some embodiments” refer to a certain feature, structure or characteristic related to at least one embodiment of the present application. Therefore, it should be emphasized and noted that, the words “an embodiment” or “one embodiment” or “an alternative embodiment” mentioned twice or more in different places of this specification do not necessarily mean the same embodiment. Moreover, some features, structures or characteristics in one or more embodiments of the present application may be combined appropriately.

Additionally, those skilled in the art can understand that, all aspects of the present application can be illustrated and described by means of several patentable categories or situations, including any new and useful processes, machines, products or combinations of substances, or any new and useful improvements thereto. Accordingly, various aspects of the present application can be completely executed by hardware, completely executed by software (including firmware, resident software, microcode, etc.), or completely executed by a combination of hardware and software. All the hardware or software mentioned above can be referred to as “data block”, “module”, “engine”, “unit”, “component” or “system”. Moreover, various aspects of the present application may be represented as computer products in one or more computer-readable media, which products include computer-readable program codes.

Unless otherwise defined, all terms (including technical and scientific terms) used here have the same meanings as those commonly understood by those of ordinary skill in the field to which the present invention belongs. It should also be understood that, terms such as those defined in common dictionaries should be interpreted as having meanings consistent with their meanings in the context of the related art, and should not be interpreted in idealized or extremely formal meanings, unless explicitly defined as such here.

The above is an explanation of the present invention, but it should not be considered as a limitation. Although several exemplary embodiments of the present invention have been described, those skilled in the art will easily understand that many modifications can be made to the exemplary embodiments without departing from the novel teaching and advantages of the present invention. Therefore, all these modifications are intended to be included in the scope of the present invention as defined in the claims. It should be understood that the above is an explanation of the present invention, and should not be considered as limited to the specific embodiments as disclosed, and modifications to the embodiments of the present disclosures and other embodiments are intended to be included in the scope of the appended claims. The present invention is defined by the claims and their equivalents.

1. An automatic detection method for detecting a content of a substance composition in a viscous material, comprising:
performing a detecting process for the viscous material in a preset detection period in order to obtain a signal detection value corresponding to the viscous material, and generating a signal detection waveform;
determining a detection state corresponding to the signal detection waveform according to the signal detection waveform, the detection state comprising a normal detection state and an abnormal detection state; and
determining the content of the substance composition in the viscous material based on the signal detection waveform only if the detection state is the normal detection state,
wherein the substance composition in the viscous material is a composition of at least one impurity substance in the viscous material; and
wherein the automatic detection method further comprises:
determining a waiting interval between a next detection and a current detection according to the detection state;
wherein the waiting interval is a first waiting interval if the detection state is determined to be the abnormal detection state, and the waiting interval is a second waiting interval if the detection state is determined to be the normal detection state, and
wherein the first waiting interval is smaller than the second waiting interval.

2. The automatic detection method according to claim 1, wherein the performing a detecting process for the viscous material in a preset detection period, in order to obtain a signal detection value corresponding to the viscous material and generating a signal detection waveform comprises:
performing a detection at a preset detection interval in the preset detection period by using a sensor detection circuit to obtain a plurality of signal detection values corresponding to the viscous material, wherein the plurality of signal detection values are values of the same type recorded at different points in time; and
performing a fitting process based on the plurality of signal detection values to obtain the signal detection waveform.

3. The automatic detection method according to claim 1, wherein the determining a detection state corresponding to the signal detection waveform according to the signal detection waveform comprises:
determining a number of valleys in the signal detection waveform; and
determining the detection state based on the number of valleys.

4. The automatic detection method according to claim 3, wherein the determining the detection state based on the number of valleys comprises:
in a case where only a single valley exists in the signal detection waveform, determining that the detection state is the normal detection state;
in a case where exactly two valleys exist in the signal detection waveform, calculating an absolute value of a difference between valley values of the two valleys, determining the detection state as the normal detection state if the absolute value of the difference is greater than or equal to a preset threshold; and determining the detection state as the abnormal detection state if the absolute value of the difference is less than the preset threshold; and
in the case where three or more valleys exist in the signal detection waveform, sorting the valley values of the valleys from smallest to largest to obtain a valley sequence, calculating an absolute value of a difference between a first valley and a second valley in the valley sequence, comparing the absolute value of the difference with a preset additional threshold, determining the detection state as the normal detection state if the absolute value of the difference is greater than or equal to the preset additional threshold; and determining the detection state as the abnormal detection state if the absolute value of the difference is less than the preset additional threshold.

5-6. (canceled)

7. The automatic detection method according to claim 1, wherein the viscous material is a viscous material arranged on a target component of a fan, and a sensor detection circuit receives the viscous material on the target component of the fan, and wherein,
in the first waiting interval, continuing to receive the viscous material by the sensor detection circuit; and
in the second waiting interval, blowing the viscous material received by the sensor detection circuit away from the sensor detection circuit and then continuing to receive the viscous material.

8. The automatic detection method according to claim 1, wherein the determining a content of the substance composition in the viscous material based on the signal detection waveform only if the detection state is the normal detection state comprises:
selecting a valley with a smallest valley value in the signal detection waveform as a target valley, and determining a detection valley value based on the valley value of the target valley, wherein the valley value is a signal value corresponding to the valley;
determining a detection environment parameter of the viscous material, the detection environment parameter comprising at least one of a detection temperature and a detection humidity; and
inputting the detection environment parameter and the detection valley value into a machine learning model, and processing the detection environment parameter and the detection valley value by the machine learning model to obtain the content of the substance composition in the viscous material.

9. The automatic detection method according to claim 8, wherein the determining a detection valley value based on a valley value of the target valley comprises:
acquiring a preset number of additional signal detection waveforms with the normal detection state;
determining a valley with the smallest valley value in each of the preset number of signal detection waveforms as the target valley; and
weighting and averaging the valley values of the target valleys of the preset number of additional signal detection waveforms to obtain the detection valley value.

10. An automatic detection device for detecting a content of a substance composition in a viscous material, wherein the automatic detection device realizes a detection of the content of the viscous material according to the automatic detection method for detecting a content of a substance composition in the viscous material according to claim 1.
